(19) United States
(12) Patent Application Publication
(10) Pub. No.: US 2024/0049952 A1
(43) Pub. Date: Feb. 15, 2024

(54) DEVICES, SYSTEMS, AND METHODS FOR TREATING KIDNEY STONES

(71) Applicant: The Regents of the University of Michigan, Ann Arbor, MI (US)

(72) Inventors: JEFFREY PLOTT, Ann Arbor, MI (US); KHURSHID GHANI, Ann Arbor, MI (US)

(21) Appl. No.: 18/034,016

(22) PCT Filed: Oct. 29, 2021
(86) PCT No.: PCT/US21/57293

§ 371 Date: Apr. 26, 2023

Related U.S. Application Data
(60) Provisional application No. 63/108,044, filed on Oct. 30, 2020.

(51) Int. Cl. A61B 1/015; A61M 1/00; A61M 3/02; A61B 1/00

(52) U.S. Cl. CPC A61B 1/015; A61M 1/80; A61M 1/77; A61M 3/0202; A61B 1/00055; A61B 1/00006; A61B 1/00057; A61M 2210/1082; A61B 1/307

(57) ABSTRACT

Provided herein are devices, systems, and methods for treating kidney stones. In particular, provided herein are irrigation and pump systems for use with endoscopic (e.g., ureteroscope) devices, and related methods for use in treating kidney stones and other applications.

### CLAIM OF PRIORITY

This application claims priority to U.S. Provisional Application No. 63/108,044, filed Oct. 30, 2020; the entire contents of which are hereby incorporated by reference.

### FIELD

Provided herein are devices, systems, and methods for treating kidney stones. In particular, provided herein are irrigation and pump systems for use with endoscopic (e.g., ureteroscope) devices, and related methods for use in treating kidney stones and other applications.

### BACKGROUND

When performing laser lithotripsy for kidney stone removal, particularly when using laser settings designed to break the kidney stone into very small particles/fragments (known in the field as “dusting”), lack of vision can quickly become an issue since it can become difficult to navigate the ureteroscope when the surrounding fluid is full of many small kidney stone fragments, or “dust”, which can impede vision. To combat this problem, many clinicians opt to use higher irrigation flow rates to help clear their field of view from small stone fragments and improve their vision. However, with higher irrigation flowrates there is a greater risk for elevated pressures in the kidney, which can lead to complications.

Thus, it is preferable to have a device/system which can maximize vision while at the same time working to ensure that fluid pressures in the kidney do not become excessively high (typically defined as >40 mmHg).

Accordingly, a need exists for improved methods of fluid management of treating kidney stones.

### SUMMARY

Provided herein are devices, systems, and methods for treating kidney stones. In particular, provided herein are irrigation and pump systems for use with endoscopic (e.g., ureteroscope) devices, and related methods for use in treating kidney stones and other applications.

The systems and methods of the present disclosure solve the limitations of existing systems for ablating kidney stones by providing flowrate and/or pressure-controlled irrigation and suction along with automated clog detection and remediation. The systems also provide customized flowrate calibration in order to prevent excessive pressure and corresponding kidney damage.

For example, in some embodiments, provided herein is a system, comprising one or more of: a) an endoscope; b) a suction component; c) an irrigation component; and d) a clog detection and removal component. In some embodiments, the system further comprises a fluid collection system (e.g., canister). In some embodiments, the suction and irrigation component are flowrate and/or pressure controlled.

The present disclosure is not limited to particular suction components. In some exemplary embodiments, the suction component comprises a volumetric peristaltic pump. In some embodiments, the flowrate controlled suction component is configured to provide suction at a flowrate between 0-40 ml/minute, although higher flow rates are contemplated.

The present disclosure is not limited to particular irrigation components. In some exemplary embodiments, the irrigation component is a pressure and/or flowrate-controlled irrigation component (e.g., including but not limited to a pressure chamber, a gravity bag, a pressure bag, or a combination thereof). In some embodiments, the irrigation component is configured to provide irrigation at a flowrate between 0-60 ml/minute.

The present disclosure is not limited to particular clog detection and removal components. In some embodiments, the clog detection and removal component comprises one or more clog detectors (e.g., a weight scale or load cell that measures the rate change in weight of a collection canister, a flowmeter in a part of an aspiration tube, an ultrasonic wave flowmeter, a photo resistor shining light through the aspiration tubing, or a pressure monitor). In some embodiments, the clog detectors are located downstream from the suction component, upstream of the suction component, within a fluid collection canister, below the fluid collection canister, after the volumetric pump, before the volumetric pump, within the fluid collection canister, below the collection canister, within the endoscope, or a combination thereof. In some embodiments, the detection and removal component further comprises a clog removal component. For example, in some embodiments, the clog removal component is configured to respond to the presence of a clog by taking one or more actions selected from, for example, reducing the irrigation pressure/flowrate to minimize a rise in intra-renal pressure; momentarily stopping/reversing the flow of the suction channel to unclog any fragments that clogged the tip of the device; momentarily increasing the flowrate of irrigation to unclog any fragments that clogged the tip of the device; alarming a user; and vibrating or actuating the endoscope to shake loose the clog. In some embodiments, the clog detection and removal component is automated or manually controlled. In some embodiments, the clog detection and removal component monitors the number of clogs and/or the severity of the clogs over time. In some embodiments, if the number and/or severity of the clogs is above a preset threshold, the system alerts a user and/or decreases the normal suction flowrate.

In some embodiments, the system further comprises an irrigation and suction flowrate calibration component. For example, in some embodiments, the irrigation and suction flowrate calibration component is configured to a) measure intrarenal pressure during and optionally before irrigation and/or suction; b) extrapolate the intrarenal pressure to determine the anticipated steady state pressure (e.g., by fitting an equation to the intrarenal pressure over time); and c) predict optimal suction and/or irrigation rates. In some embodiments, the irrigation and suction flowrate calibration component is further configured to adjust input pressure and/or flowrate to keep steady state intrarenal pressure below a set threshold level. In some embodiments, the irrigation and suction flowrate calibration component comprises a pressure sensor (e.g., a pressure sensor wire).

The present disclosure is not limited to particular endoscope devices. In some embodiments, the endoscope is a ureteroscope. In some embodiments, the endoscope is an endoscopic device comprising a distal end, the distal end comprising: a) a first channel configured for delivery of fluid; and b) a second channel configured to remove fluid via suction (e.g., wherein the second channel exits the distal end on a different plane than the first channel). In some embodiments, the exit of the first channel or the second channel comprises a suction port. In some embodiments, at least one of the first and second channels are configured to prevent stones from occluding the suction port. In some embodiments, the exit of the first channel and/or the exit of the second channel is substantially planar and/or substantially in the first and/or second plane. In some embodiments, the endoscopic device comprises an outer housing surrounding an interstitial space, wherein the endoscopic device comprises at least one interstitial flow opening(s) in fluid communication with the interstitial space, wherein the interstitial flow opening(s) are configured to deliver fluid or suction through the interstitial space; and a fluid port. In some embodiments, the endoscope is an endoscopic device, comprising: an outer housing surrounding an interstitial space, wherein the endoscopic device comprises at least one interstitial flow opening(s) configured to deliver fluid or suction through the interstitial space.

Further embodiments provide a system, comprising: a) an endoscope comprising a distal end, the distal end comprising: a) a first channel configured for delivery of fluid; and b) a second channel configured to remove fluid via suction (e.g., wherein the second channel exits the distal end on a different plane than the first channel) and wherein the exit of the second channel comprises a suction port; b) a flowrate and/or pressure controlled suction component and/or a flowrate and/or pressure controlled irrigation component; and c) an irrigation and/or suction flowrate calibration component configured to i) measure intrarenal pressure during and optionally before irrigation and/or suction; ii) extrapolate the intrarenal pressure to determine the anticipated steady state pressure (e.g., by fitting an equation to the intrarenal pressure over time); and iii) predict optimal irrigation and/or suction parameters that maintain steady state intrarenal pressure at or below a threshold level.

Additional embodiments provide a system, comprising: a) an endoscope comprising a distal end, the distal end comprising: i) a first channel configured for delivery of fluid; and ii) a second channel configured to remove fluid via suction (e.g., wherein the second channel exits the distal end on a different plane than the first channel and wherein the exit of the second channel comprises a suction port); b) a flowrate and/or pressure controlled suction component and/or a flowrate and/or pressure controlled irrigation component; and c) a clog detection and removal component.

Yet other embodiments provide a method of ablating a kidney stone, comprising: a) introducing the endoscopic device of a system described herein into the ureter of a subject; b) advancing the endoscopic device to a kidney stone while the flowrate controlled suction component, the irrigation component, and the clog detection and removal component are active; and c) ablating the stone using the endoscopic device.

Still other embodiments provide the use of a system described herein (e.g., for the removal of a kidney stone).

In further embodiments, provided herein is a method of detecting and removing a clog during removal of a kidney stone, comprising: a) introducing the endoscopic device of a system described herein into the ureter of a subject, and advancing the endoscopic device to a kidney stone; and b) ablating the kidney stone while the clog detection and removal component i) monitors the system for a potential clog; ii) detects the clog; and iii) removes the clog.

In certain embodiments, provided herein is a method of calibrating the suction flowrate and/or pressure of a system described herein, comprising: a) introducing the endoscopic device of a system described herein into the ureter of a subject, and advancing the endoscopic device to a kidney stone; b) ablating the kidney stone while the clog detection and removal component i) monitors the system for a potential clog; ii) detects and removes any clogs that are present; c) records the rate and/or the severity of clogs detected; and d) adjusts the suction flowrate/pressure to either i) reduce the suction flowrate and/or pressure to reduce the rate and/or the severity of future clogs if the clog rate and/or severity is deemed too large or ii) increase the suction flowrate and/or pressure if no clogs or a level of clogs below a certain threshold amount of clogs and/or severity have occurred in a given time period.

In additional embodiments, provided herein is a method of calibrating irrigation and/or suction flowrate during removal of a kidney stone, comprising: a) introducing the endoscopic device of a system described herein into the ureter of a subject, and advancing the endoscopic device to a kidney stone; b) measuring intrarenal pressure during and optionally before irrigation and/or suction; c) extrapolating the intrarenal pressure to determine the anticipated steady state pressure (e.g., by fitting an equation to the intrarenal pressure over time); and d) predicting optimal irrigation and/or suction parameters that maintain steady state intrarenal pressure below a threshold level.

Additional embodiments are described herein.

### DESCRIPTION OF THE DRAWINGS

FIG. 1 shows an overview of exemplary suction and irrigation system of embodiments of the present disclosure.

FIG. 2 shows a flow chart for exemplary clog detection and mitigation systems of embodiments of the present disclosure.

FIG. 3 shows a flow chart for exemplary irrigation flow rate calibration systems of embodiments of the present disclosure.

FIG. 4 shows a flow chart for exemplary irrigation and suction flow rate calibration systems of embodiments of the present disclosure.

FIG. 5 shows a graph of pressure over time using an exemplary irrigation flow rate calibration system of embodiments of the present disclosure.

FIG. 6 shows a graph of pressure over time using an exemplary irrigation flow rate calibration system of embodiments of the present disclosure.

FIGS. 7A and 7B show exemplary endoscopes for use in the systems of embodiments of the present disclosure.

FIGS. 8A and 8B show exemplary endoscopes for use in the systems of embodiments of the present disclosure.

FIG. 9 shows an exemplary system of embodiments of the present disclosure.

FIG. 10A-C show an exemplary endoscope for use in the systems of embodiments of the present disclosure.

FIG. 11 shows a flow chart for exemplary clog detection and mitigation systems of embodiments of the present disclosure.

### DESCRIPTION

Provided herein are devices, systems, and methods for treating kidney stones. In particular, provided herein are irrigation and pump systems for use with endoscopic (e.g., ureteroscope) devices, and related methods for use in treating kidney stones and other applications.

Suction in combination with irrigation has been tried to help enable increased irrigation flowrates while simultaneously mitigating elevated pressures within the kidney. However, due to the high concentration of kidney stone fragments/particles, there is the potential for the suction channel of a device to become clogged. Further, systems have been contemplated which can detect a clog in the suction system and can alert the user that such a clog has occurred (see, U.S. Pat. No. 9,883,885). However, especially in the case of kidney stone laser lithotripsy surgery, simply being alerted to the fact that a clog has occurred comes with the limitation that the clog still exists and will require intervention from the clinician in order to remedy the clog. Further, others have contemplated strategies of remedying a clog by articulating the suction/vacuum lumen to remove any kinks, further increasing the suction to de-clog the vacuum lumen, or temporarily placing the vacuum lumen in fluid communication with a positive pressure source (e.g., a syringe) which may be temporarily turned off or disconnected to force fluid distally through the vacuum lumen to dislodge the obstruction, or using an unclogging obturator in the vacuum lumen (see, WO2019152727). However, these methods of lumen unclogging require intervention from the user that can lead to reduced efficiency in the operating room.

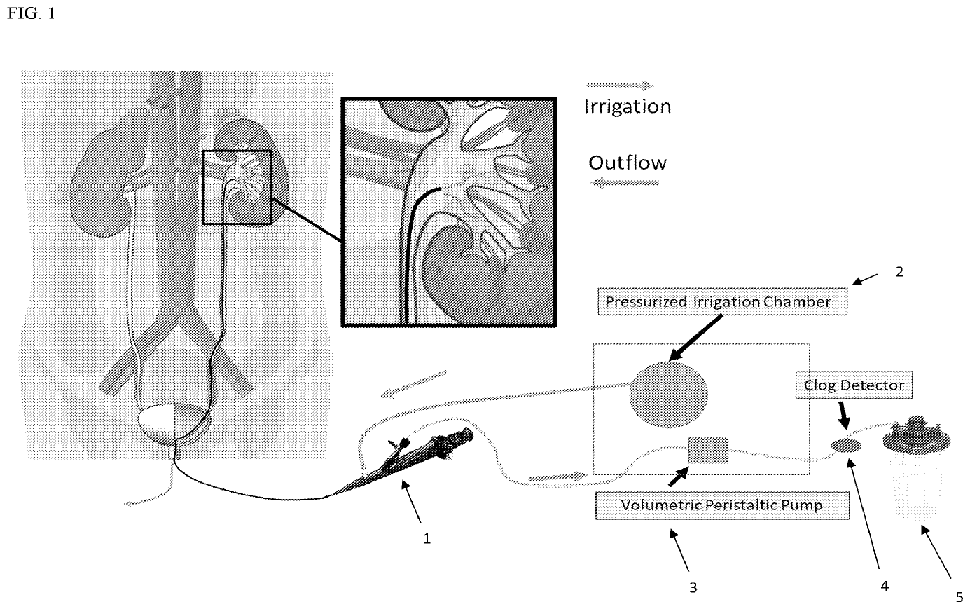

The present disclosure addresses these limitations of existing systems by providing, for example, an irrigation and suction system comprising one or more of a) an endoscope; b) a suction component; c) an irrigation component, and d) a clog detection and removal component. FIG. 1 shows an exemplary system of embodiments of the present disclosure. In some embodiments, as shown in FIG. 1, the system comprises an endoscope 1 (shown as a ureteroscope in FIG. 1), a pressurized irrigation component 2 (shown as a pressurized irrigation chamber in FIG. 1), suction component 3 (shown as a volumetric peristaltic pump in FIG. 1), and a clog detection component 4 (shown as a clog detector in FIG. 1). Also shown in FIG. 1 is fluid collection canister 5 for collection of waste fluid. In some embodiments, systems further comprise an irrigation and/or suction flow rate calibration component (not shown in FIG. 1).

Each of the components is discussed in detail below.

The present disclosure is not limited to the suction and irrigation components shown in FIG. 1. Any suitable suction and irrigation components may be utilized. In some embodiments, the pressurized irrigation component is a peristaltic pump, pressure chamber, gravity bag, pressure bag, or combination. In some embodiments, the irrigation component provides a variable flowrate between 0-60 ml/min in increments of at least 5 ml/min. For example, in some embodiments, the irrigation flowrate is approximately 20 ml/min (e.g., plus or minus 1%, 5%, 10%, 15%, or 20%).

The optimal pressure is based on the flowrate and varies based on the particular endoscope utilized. In some embodiments, experiments conducted during development of the present disclosure determined that pressure between 0-600 cmH₂O provides adequate interstitial flow in an 8.5 Fr scope.

If using pressure control, the pressure will give you a resultant flowrate. The pressure may be controlled between, say 0-500 mmHg, which will give you resultant flowrates that are clinically desirable. The base pressure is also resultant on the system that it is pushing fluid through so that pressure range may need to be different depending the on the endoscope design and the system utilized.

In some embodiments, the suction component is a volumetric peristaltic pump. In some embodiments, the suction component is a vacuum based system (e.g., that monitors the suction flowrate to adjust the pressure accordingly), a progressive cavity pump, a rotary pump, a piston pump, a gear pump, an infusion pump (e.g., and the like. Peristaltic is the most medically relevant type though for this application. Infusion pump: https://www.unilever-medical.com/shop/wif-medical-wif-302-pump-iv-infusion).

In some embodiments, fluid is contained within a flexible tube fitted inside a circular pump casing (though linear peristaltic pumps may be used). A rotor with a number of rollers, shoes, wipers, or lobes attached to the external circumference of the rotor compresses the flexible tube. As the rotor turns, the part of the tube under compression is pinched closed (or occludes) thus forcing the fluid to be pumped to move through the tube. Additionally, as the tube opens to its natural state after the passing of the cam (“restitution” or “resilience”) fluid flow is induced to the pump.

In some embodiments, the suction component provides a variable flowrate between at least 0-40 ml/min in increments of at least 2 ml/min. For example, in some embodiments, the suction flowrate is approximately 10 ml/min (e.g., plus or minus 1%, 5%, 10%, 15%, or 20%). In some embodiments, higher momentary suction flowrates (˜60 ml/min) are potentially desirable for stone repositioning.

In some embodiments, the suction component provides a controlled suction rate. Having a controlled suction rate is useful because the rate of suction flowrate can influence the size of stone particles that are attracted into the suction channel. For example, a higher volumetric suction flowrate pulls in larger size stone fragments (which are more likely to clog a suction channel) while a lower volumetric suction flowrate tends to only pull in relatively smaller stone fragments, which are less likely to clog the suction channel. However, one generally prefers to maximize the suction flowrate so that the irrigation flowrate can be safely increased, allowing for improved vision clearing without excessively increasing the pressure within the kidney.

In some embodiments, the irrigation and suction components interface with collection and irrigation containers. In some embodiments, the irrigation and suction components are controlled via a user interface on the endoscope handle.

In some embodiments, systems of the present disclosure comprise a clog detection component comprising one or more clog detectors. In some embodiments, the clog detection system comprises multiple clog detectors located at multiple points along the system such as, for example, after the volumetric pump (as shown in FIG. 1), before the volumetric pump, within the fluid collection canister, below the collection canister, or within the ureteroscope itself.

The present disclosure is not limited to particular clog detectors. In some embodiments, the clog detector is a weight scale or load cell that measures the rate change in weight of the collection canister. When that rate change reduces, a clog may have occurred. Alternative examples include but are not limited a flowmeter in a part of the aspiration tube, an ultrasonic wave flowmeter, a pressure monitor connected to the line, or other methods that detect a clog in the system.

In some embodiments, the system comprises an automated or manual clog response component. If the clog sensor detects there is a clog, the system can respond in several ways to clear the clog. For example, in some embodiments, the system responds to a clog by (1) reducing the irrigation pressure/flowrate to minimize a rise in intra-renal pressure, (2) momentarily stopping/reversing the flow of the suction channel to unclog any fragments that clogged the tip of the device, (3) alarming the clinician, (4) vibrating or actuating any working channel instrumentation (e.g., laser fiber) to shake loose the clog, or (5) a combination thereof. In some embodiments, flow reversal is automatically initiated upon detection of a clog. The flow reversal can be for a sufficiently short period of time (e.g., less than 1 second, less than 0.5 seconds, less than 0.1 seconds) so as to not interfere with a live procedure.

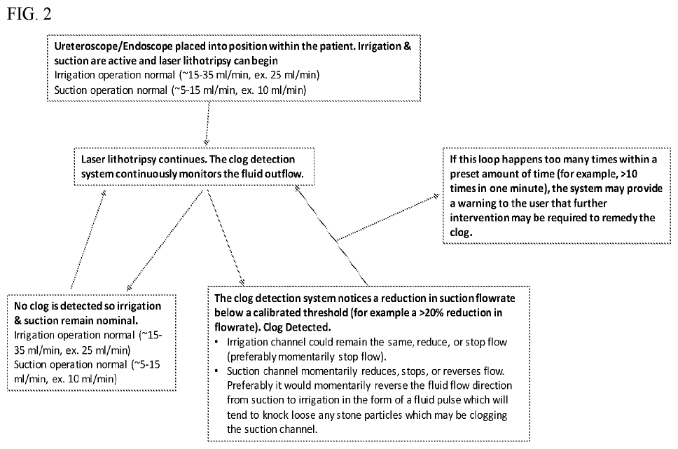

FIG. 2 shows an exemplary block diagram for a clog detection and response component in use. An endoscope is placed in position in a patient with the irrigation and suction components in use and operating at a normal flow rate. The clog detection component monitors the fluid flow during the procedure. If a clog is detected (e.g., via a reduction in suction flowrate below a calibrated threshold or other method), the system responds (e.g., in an automated manner via software control or manually by a user) by taking one of several actions (e.g., those described above or a reduction in the suction flowrate) in order to dislodge particles clogging the endoscope. In some embodiments, the clog detection and response component automatically detects and removes clogs before a user observes or detects the clog, thus saving time and avoiding a potential distraction.

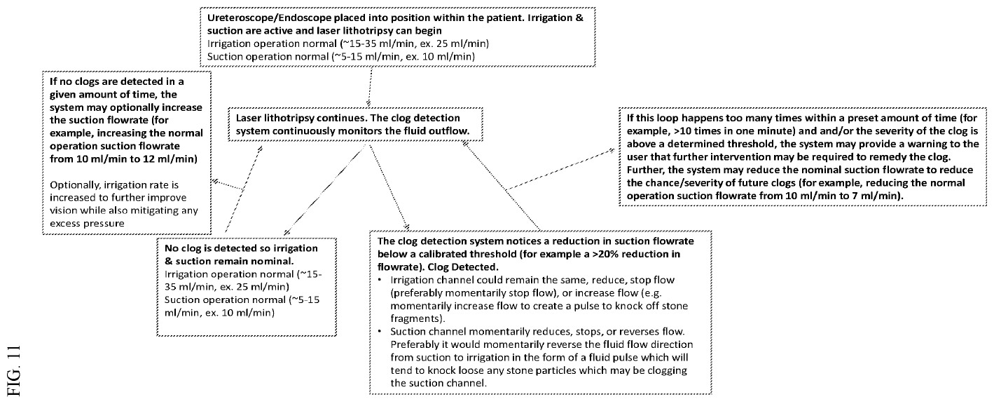

FIG. 11 shows an exemplary block diagram for a further embodiment of a clog detection and response component in use. In some embodiments, the clog detection and response component monitors the number of clogs detected and removed over time, as well as the severity of the clog. If the number of clogs is above a preset threshold, and/or the severity of the clogs is above a present threshold, the system takes a further action (e.g., provides a warning to the user and/or a reduction in the normal operation suction flowrate. In some embodiments, if no clogs are detected in a given time period, the system optionally increases the suction and optionally irrigation flowrate.

In some embodiments, systems described herein further comprise an irrigation and/or suction flowrate calibration system to optimize the irrigation and suction flowrate for a particular endoscope and/or subject. Each kidney and ureter system has a different level of compliance. Compliance is defined as the ease of passive outflow of irrigation fluid from the kidney/ureter system. Compliance of the kidney is also a term to describe its elastic distensibility. Compliance also describes the relationship between change in volume and change in pressure. Poor compliance results in higher intra-renal pressures.

By understanding the system compliance (e.g., when using an endoscopic device either with or without an access sheath), irrigation flows are optimized such that the highest irrigation flow that can be safely used for a patient (e.g., one typically wants to keep intra renal pressures below 40 mmHg) are obtained. In general, the higher the irrigation flow, the better visualization for the clinician, leading to more efficient procedures.

Further, pressure-based irrigation systems are commonly used since they will automatically tend to reduce the irrigation flowrate as the intrarenal pressure increases, providing a built-in safety measure. However, since pressure based irrigation flowrate is also dependent on the resistance of the ureteroscope fluid path, since the irrigation is passed into the kidney through the ureteroscope, it can be difficult to control when providing irrigation through the working channel of the ureteroscope since a variety of different instrumentation may be placed in the working channel throughout the procedure (e.g., laser fibers, baskets, biopsy forceps, nothing). All these potential scenarios can change the irrigation inflow resistance and thus the irrigation flowrate that may be achieved at a given irrigation input pressure.

For example, prior data in a kidney simulation model shows that a compliant system achieves a steady state intra renal pressure of 37 mmHg with an irrigation source pressure of 225 mmHg and no suction to achieve an irrigation flowrate of 20.89 ml/min. At that same irrigation source pressure, suction can be turned on at 12 ml/min to change the intra renal pressure to 14.8 mmHg with an irrigation flowrate of 21.48 ml/min. This allows the clinician to know that they will always remain in a safe intra renal pressure situation whether or not the suction channel is clogged by debris.

Further, in a kidney simulation model setup as a non-compliant system (no passive outflow), an irrigation source pressure of 150 mmHg and no suction can lead to dangerously high intra renal pressures approaching 150 mmHg and minimal to no irrigation inflow. With suction turned on at 12 ml/min, the irrigation flowrate then also becomes 12 ml/min and the intra renal pressure falls to a safe 28.8 mmHg. However, since this is a non-compliant system, by increasing the irrigation source pressure to 225 mmHg (with suction on), the intra renal pressure increases to 95.8 mmHg without any improvement in irrigation flowrate. This demonstrates that in certain scenarios, higher irrigation flow pressures may not necessarily lead to better vision and can put the patient at greater risk for complications.

In some embodiments, devices utilize interstitial irrigation rather than working channel irrigation, which results in the resistance of the ureteroscope for irrigation fluid being constant throughout the entire procedure. However, there is still a benefit to customization of flow rate to a given endoscope and patient.

The present disclosure, in some embodiments, addresses these issues by providing an integrated irrigation and/or suction rate calibration component.

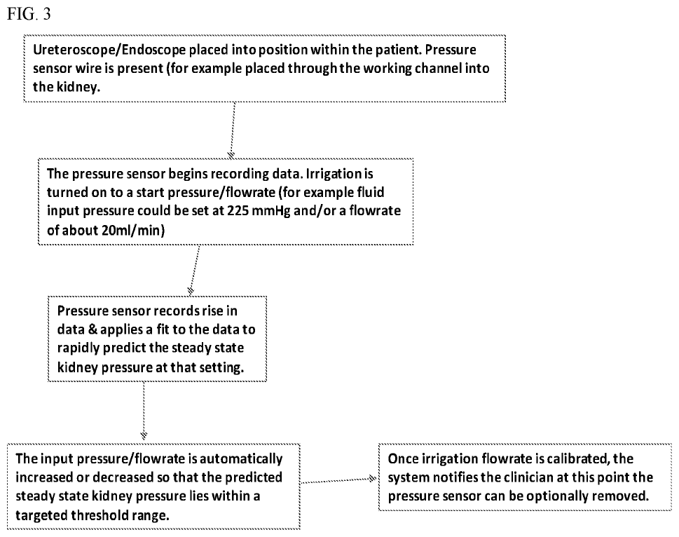

FIG. 3 shows a flow chart for an exemplary irrigation flowrate calibration component. Once the endoscope is placed in the patient, a pressure sensor is used to measure intra renal pressure. The pressure sensor is located at any suitable location on the endoscope (e.g., on the scope, detachable, built-in, passed through working channel, etc.). In endoscopes where the interstitial space is utilized, the pressure sensor can be inserted and removed from the working channel without changing the ureteroscope resistance for irrigation fluid. In such embodiments, the pressure sensor does not need to be built into the scope which can increase device size and complexity.

Initially, irrigation flowrate is started without suction. The pressure within the kidney typically starts to rise. The sensor communicates this pressure data to the pump system.

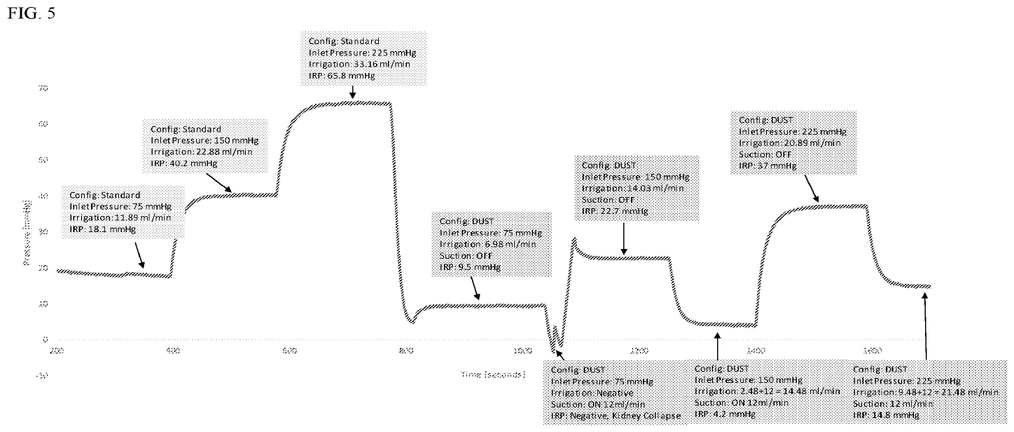

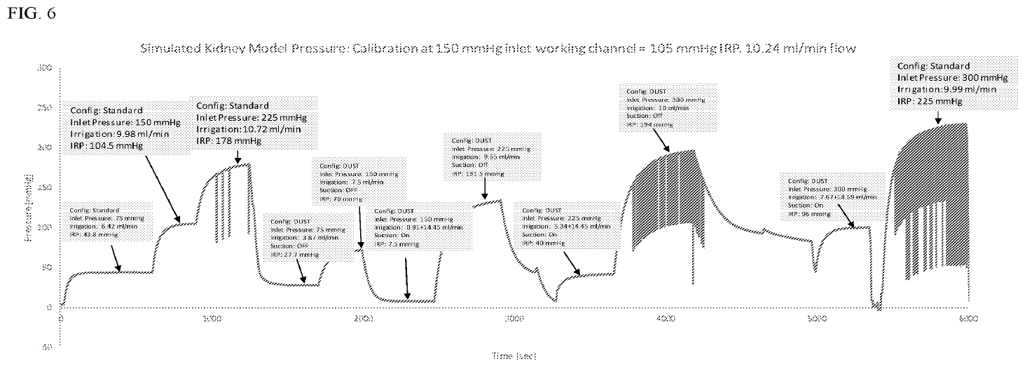

The pump system then analyzes the pressure curve to understand the compliance of the kidney system (FIGS. 5-6). FIGS. 5-6 show calibration to two different intrarenal pressure levels 40 mmHg (FIG. 5) and 104 mm Hg (FIG. 6) in a 3.6 Fr working channel device at 150 mmHg inlet pressure with a 200 Flexiva™ laser fiber (Boston Scientific) inserted in the working channel. FIGS. 5-6 shows calibration using a “standard” ureteroscope configuration where irrigation is provided through a working channel and a “dust” configuration where irrigation is provided through interstitial space.

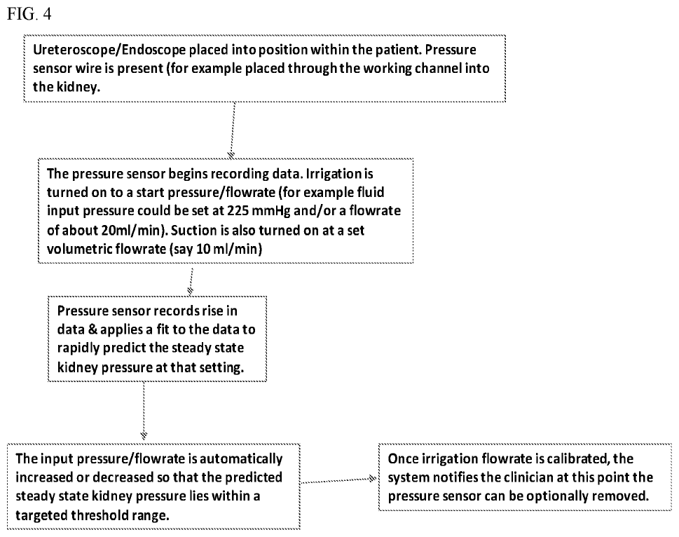

The system uses the pressure sensor data to apply a fit (e.g., equation) to the data to predict the steady state kidney pressure for the given endoscope and patient. Each type of kidney has a slope of rise, so for example, one can perform a logarithmic extrapolation after, for example, a time period (e.g., 30 s) of pressure recording, to extrapolate what the maximum intra renal pressure will be. This can be repeated with irrigation and suction on to determine the minimum intra renal pressure (assuming irrigation and suction occur at the same time) (See FIG. 4).

Then, in some embodiments, the input pressure and/or flowrate is automatically increased or decreased so that the predicted steady state kidney pressure lies within a targeted threshold range. In some embodiments, once calibration is completed, the clinician is notified, and the pressure sensor is optionally removed. The procedure is then continued.

In some embodiments, the pressure sensor is re-introduced to check calibration, as well as at end to see the pressure in kidney is safe.

In some embodiments, irrigation is provided through interstitial space, which results in a constant irrigation flowrate resistance that is not interfered by instruments in the working channel, making this a unique feature and enabling the use of a constant pressure control.

FIG. 4 shows a flow chart for an exemplary irrigation and suction flowrate calibration component. Once the endoscope is placed in the patient, a pressure sensor is used to measure intrarenal pressure. The pressure sensor is located at any suitable location on the endoscope (e.g., on the scope, detachable, built-in, passed through working channel, etc.).

Irrigation flowrate is started with suction. The pressure within the kidney typically starts to rise. The sensor communicates this pressure data to the pump system.

The pump system then analyzes the pressure curve to understand the compliance of the kidney system (FIGS. 5-6). The system uses the pressure sensor data to apply a fit to the data to predict the steady state kidney pressure for the given endoscope and patient. Then, the input pressure and/or flowrate is automatically increased or decreased so that the predicted steady state kidney pressure lies within a targeted threshold range. In some embodiments, once calibration is completed, the clinician is notified, and the pressure sensor is optionally removed. The procedure is then continued.

In some embodiments, the pressure sensor is re-introduced to check calibration, as well as at the end of the procedure to see the pressure in kidney is safe.

Not only is it important to make sure the kidney pressure does not get too high, but it also should not get too low. If needed, the system can use a different irrigation source pressure, for example, a high irrigation source pressure, when combined with suction such that the intra renal pressure remains above a lower threshold. This method allows an even greater irrigation flow rate while still maintaining a safe intrarenal pressure.

Further, the system can detect when a clog occurs in the suction system as described above. If the clog occurs, the system can revert to a safer lower irrigation source pressure (previously defined) until the suction clog is remedied.

In some embodiments, after sufficient testing is complete, it is possible for the system to work without using a pressure sensor. For example, if using a known access sheath with a known ureteroscope, it can use pre-prescribed irrigation source pressures shown to be safe in previous studies. Further, if not using an access sheath, the clinician can input a perceived compliance based on previous knowledge (for example, if the patient previously had a stent or not) to select an appropriate source irrigation pressure.

In some embodiments, the systems of the present disclosure comprise an endoscope (e.g., ureteroscope). The present disclosure is not limited to particular endoscope designs. In some embodiments, commercially available endoscopes are utilized (e.g., including but not limited to, those available from Dornier MedTech, Munich, Germany or Richard Wolf, Vernon Hills, IL).

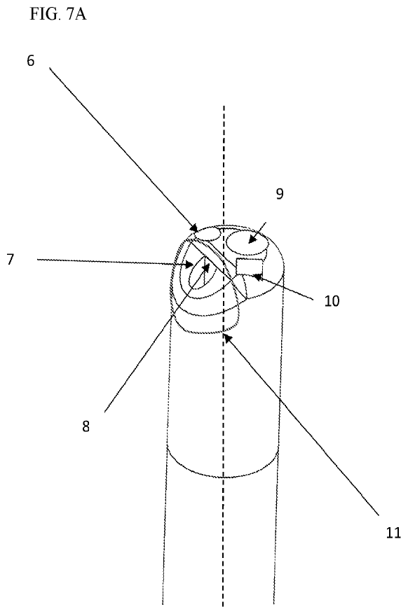

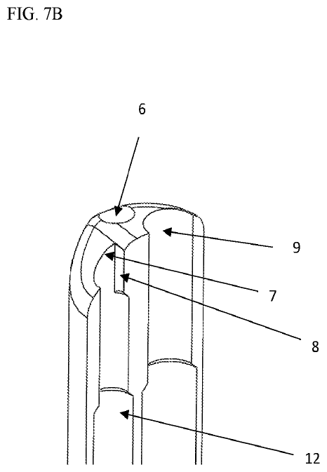

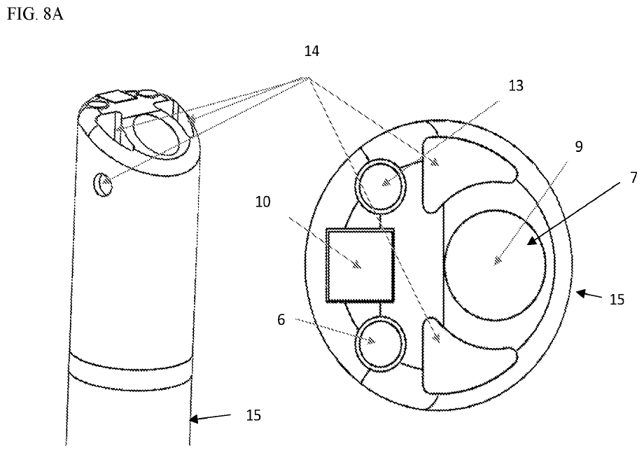

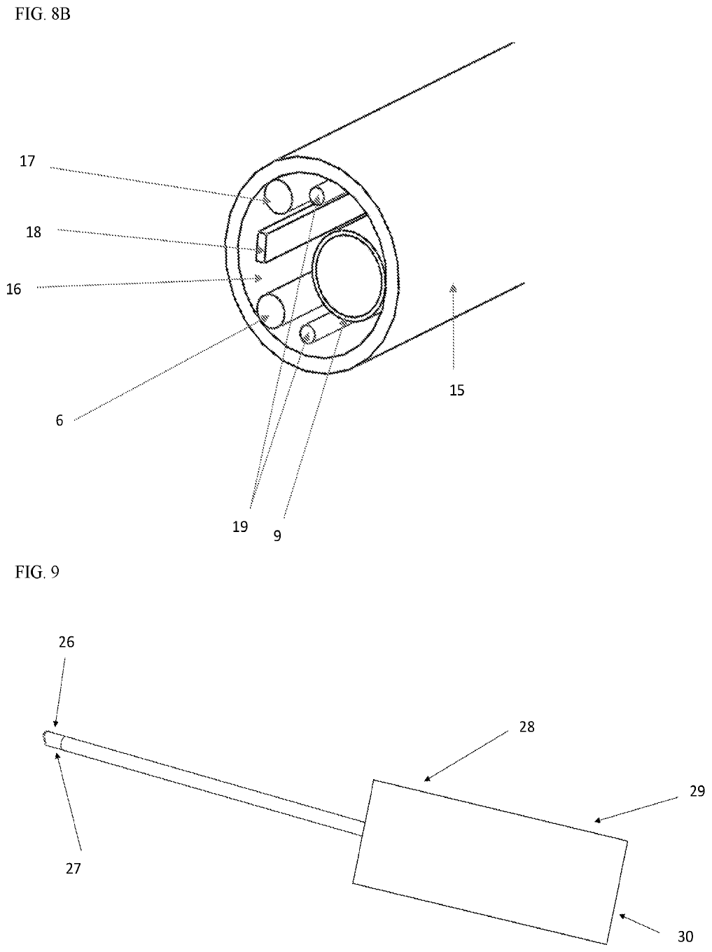

In some embodiments, the endoscopes described in PCT/US2020/030605; herein incorporated by reference in its entirety, are utilized. For example, in some embodiments, ureteroscope devices shown in FIGS. 7-8 are utilized. Referring to FIG. 7A, shown is a ureteroscope tip comprising light 6, suction port 7, anti-clog inlet or feature 8 (configured to prevent clogging of the suction port and/or suction channel by large stones or stone fragments), working (e.g., laser and/or irrigation) channel 9, camera 10, and suction region 11, which optionally may be made of compliant material. The dashed line represents the longitudinal axis of the ureteroscope. The working channel 9 is presented on the distal end of the ureteroscope tip in a plane perpendicular to the longitudinal axis of the ureteroscope. Suction port 7 is presented on a different plane that the working channel 9, for example at a 20 to 70 degree angle relative to the plane of the working channel 9 (although the disclosure is not limited to these particular dimensional relationships). In some embodiments, the suction port 7 is on a different plane from the camera 10. In some embodiments, this prevents the camera image from becoming blocked by a stone or stone fragments during fluid suction through the suction channel. As a stone or stones collect in front of the suction channel, they will tend to angle away from the camera field of view, helping to mitigate the full camera obstruction so the clinician can continue to see where they are in the kidney. In some embodiments, region 11 is not made of compliant material, but rather a non-compliant material. In the device shown in FIG. 7A, the face surrounding the suction port is flat, although other geometries are specifically contemplated. The light 6 serves to illuminate the working area.

FIG. 7B shows a cross-section cut-out view of the device of FIG. 7A. Shown is light 6, suction port 7, anti-clog inlet 8, working channel 9, and suction channel 12. The suction channel 12 and working channel continue through the device and exit the proximal end of the device (not shown). In some embodiments, the diameter of the suction channel 12 and/or working channel 9 are narrower near the distal opening as shown in FIG. 7B. This first narrowing can assist with device assembly, providing a hard stop for tubing used for the suction channel 12 and/or working channel 9. In some embodiments, this first narrowing matches the inner diameter of the tubing connected to the ureteroscope tip. In some embodiments, the tubing has metallic braiding (e.g., stainless steel or nitinol) in the wall to prevent distal channel kinking during ureteroscope articulation. An additional narrowing, such as seen with anti-clog inlet 8, can be utilized to prevent stone fragments of a certain size from entering suction channel 12. This can act to reduce the chances for suction channel 12 to clog with fragments during the procedure. In other embodiments, channel diameters are constant throughout the device.

In some embodiments, devices comprise symmetrical (e.g., comprising symmetry around the bending axis of the device) working channels 9 that interchangeably serve as suction or laser/irrigation channels. For example, in some embodiments, a user can select either channel for suction or laser/irrigation use depending on the laterality of the kidney (e.g. right or left side), region of the kidney, shape or location of stone, or other factors. This provides for improved visibility and access to stones using a single device. In some embodiments, channels are used interchangeably during a single procedure (e.g., a user switches the function of a channel during a single procedure on a single stone or multiple stones).

In some embodiments, the suction port 7 and exit of working channel 9 are each substantially in a plane or are planar. As used herein, the term “substantially in a plane” or “substantially in the plane” in reference to an opening or exit of a channel or port of a device described herein refers to an opening or exit that is at least 90% (e.g., at least 90%, 91%, 92%, 93%, 94%, 95%, 96%, 97%, 98%, 99% or 100%) in a single plane (or substantial plane).

As used herein, the term “substantially planar” when used in reference to an opening or exit of a channel or port of a device described herein refers to an opening or port that is at least 90% (e.g., at least 90%, 91%, 92%, 93%, 94%, 95%, 96%, 97%, 98%, 99% or 100%) planar throughout the entire opening or port.

Not all openings on the single device need to be in the same plane or a plane with the same angle relative to the longitudinal or perpendicular axis of the device in order to be individually substantially in a plane or planar.

In some embodiments, provided herein is a ureteroscope that utilizes the interstitial space inside an outer housing of the device to deliver irrigation fluid at or near the tip of the device while simultaneously using the working channel for fluid suction and/or other device components. This greatly improves visualization for the procedure while enabling a smaller device outer diameter. In practice, irrigation fluid can be pressurized and flow from an inlet port in the device handle, through the interstitial space within the ureteroscope outer housing, and exit the device through the one or more interstitial flow openings. In some embodiments, these openings are at the tip of the device to help clear away debris from the field of view, although the present disclosure is not limited to a particular location. Examples include, but are not limited to, on the top surface of the tip, the side surface of the tip, through an opening on the outer housing, or a combination thereof.

Now referring to FIG. 8, shown is a device configuration that utilizes an outer housing and interstitial fluid openings. In some embodiments, this configuration utilizes cutouts (e.g., interstitial flow openings) placed at or near the tip (e.g., distal end) of the device to direct pressurized irrigation fluid into the kidney without disturbing the small stone fragments (e.g., improving popcorn lithotripsy efficiency). In some embodiments, this configuration utilizes cutouts (e.g., interstitial flow openings) placed at or near the tip of the device to direct pressurized irrigation fluid into the kidney to clear the small stone fragments (e.g., improving visualization). The outer housing serves as a pseudo-working channel for fluid delivery or suction. Herein, a pseudo-working channel is a channel that allows for fluid delivery and/or suction but cannot accommodate instrumentation exchanges such as passing a laser fiber or basket through the channel during the procedure since there is not a direct channel or tube connecting the interstitial flow opening(s) to a port outside of the patient (e.g. in the device handle). In some embodiments, irrigation fluid is pumped in between the inner surface of an outer housing and the outer surface of an inner working channel (e.g., via a fluid port). In some embodiments, fluid is removed through the interstitial space between the outer housing and the inner working channel (e.g., via a suction port). In some embodiments, both irrigation and suction are interchangeably applied through the interstitial space between the outer housing and the inner working channel. In some embodiments, the interstitial space and the working channel are substantially (e.g., completely or partially) fluidly separate.

FIG. 8A shows perspective (left panel) and top (right panel) views of a device comprising interstitial flow openings 14. FIG. 8 shows two interstitial flow openings 14 located on the distal end of the device and one on the side. However, the present disclosure is not limited to such a number or configuration of interstitial flow openings 14. For example, in some embodiments, devices comprise one or more (e.g., 1, 2, 3, 4, 5, or more) interstitial flow openings 14. The interstitial flow openings are placed in any suitable location including but not limited to, on the tip or side of the device. FIG. 8A further shows outer housing 15, light 6, working channel 9, camera 10, and pressure sensor 13.

FIG. 8B shows a cut-out view of the tip of the device shown in FIG. 8A. FIG. 8B illustrates an outer housing 15 surrounding interstitial space 16. As described above, in some embodiments, the interstitial space 16 serves as a conduit to deliver irrigation to the working field (e.g., through interstitial openings (shown in FIG. 8A) or to provide suction. The interstitial space 16 further provides a location for device components such as, for example, including but not limited to, pressure sensor wire 17, camera wire 18, and pull wires 19 (e.g., articulation pull wires). In some embodiments, interstitial space 16 further provides a location for articulation elements to allow the navigation of the tip via pull wires 19. Also shown is working channel 9 and wire for light 6.

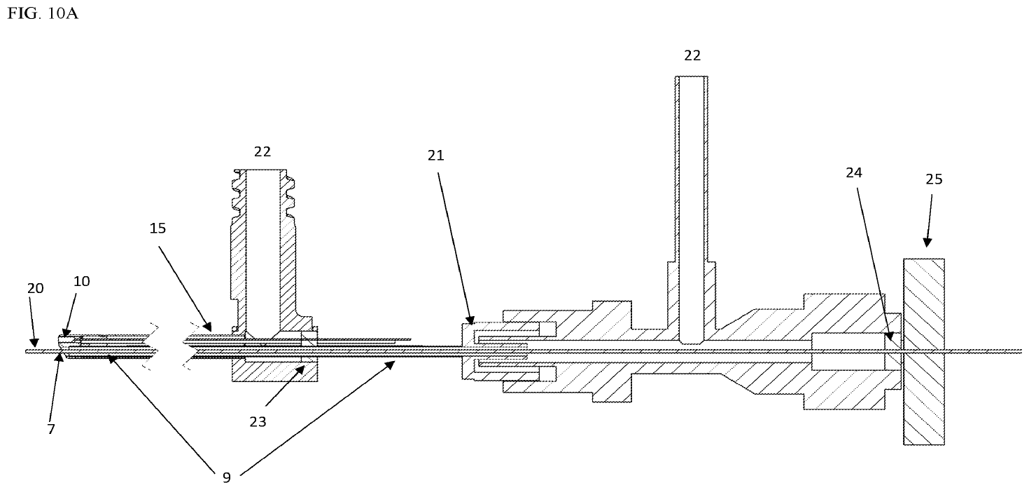

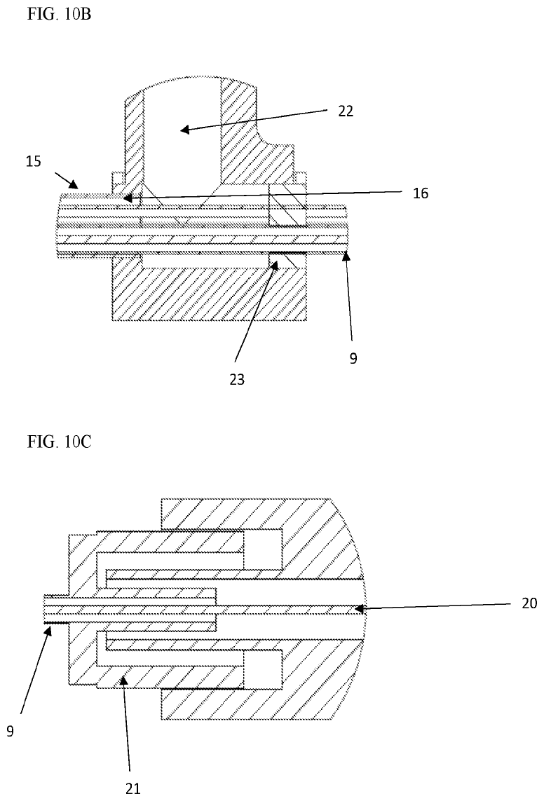

FIG. 10A-C shows an embodiment comprising a device that utilizes the interstitial space for a variety of functions.

Now referring to FIG. 10A, shown is a section view of an exemplary device comprising an outer housing 15 and interstitial flow openings (not shown in FIG. 10A). Shown are working channel 9, suction port 7, laser 20, suction connection 21, and camera 10. Also shown in FIG. 10A is one or more fluid ports 22. The one or more fluid ports 22 are located at any suitable or convenient location on the device (e.g., the handle (not shown) or other portion of the proximal (e.g., handle) or distal (e.g., tip) end). In some embodiments, devices comprise one or more fluid ports 22 that are in fluid communication with the working channel 9 and/or the interstitial space. For example, in some embodiments (e.g., the left fluid port 22 shown in FIG. 10A) the fluid port 22 is in fluid communication with the interstitial space 16 (not shown in FIG. 10A). The fluid port 22 provides an inlet to provide fluid and/or suction at or near the tip of the device via fluid port 22. In some embodiments, the fluid port (e.g., the right fluid port 22 shown in FIG. 10A) is in fluid communication with working channel 9.

Still referring to FIG. 10A, in some embodiments, the internal components are sealed to allow irrigation to flow through the device without damaging any internal components of the device. For example, in some embodiments, a fluid port 22 in fluid communication with the interstitial space comprises a fluid seal 23 between the fluid port 22 and the outer diameter of working channel 9. Pull wires, camera wire, light, and sensor (not shown in FIG. 10A), and other components can pass through this seal. The seal prevents fluid in the interstitial space from flowing into the handle of the endoscope/ureteroscope device and focuses the fluid pressure toward interstitial flow opening(s). Fluid seal 23 can be composed of multiple suitable materials such as, for example but not limited to, conformable elastomeric element(s), adhesive resin, adhesive resin with internal channels and sealant, or other combinations thereof. Some elements passing through fluid seal 23, such as a camera wire, may not need to translate and thus are glued/sealed into place at fluid seal 23. Other elements passing through fluid seal 23, for example pull wires for device articulation, may need to repeatedly translate proximally and distally with respect to fluid seal 23. In that scenario, it may be preferable to utilize, for example, elastomeric elements and/or a tubing channel with tight tolerance to the pull wire(s) and optionally a sealing lubricant (e.g., medical grade silicone grease), to generate a fluid tight seal that still allows translation of select components about the fluid seal 23. In some embodiments, a fluid port 22 in fluid communication with working channel 9 comprises laser fiber seal 24. In some embodiments, laser fiber seal 24 provides a fluid seal between the laser 20 and working channel 9. This focuses the vacuum pressure at the fluid port 22 to pull fluid through the suction port opening, through the working channel 9, and into a fluid collection tank (not shown in FIG. 10). In some embodiments, laser fiber seal 24 is composed of an elastomeric element and can be selectively loosened or tightened to allow repositioning of the laser 20.

Still referring to FIG. 10A, shown is laser slider 25. In some embodiments, laser slider 25 is used to optionally linearly actuate the laser fiber (e.g., in the plane of the ureteroscope). This can help unclog any stone fragments from the working channel 9 that may potentially build up and limit the suction flow.

FIG. 10B shows a close-up view of fluid seal 23. Fluid seal 23 fluidly isolates working channel 9 from the labeled fluid port 22. In FIG. 10B, fluid port 22 is in fluid communication with interstitial space 16 and is fluidly sealed to outer housing 15.

FIG. 10C shows a close-up view of suction connection 21. Suction connection 21 fluidly connects working channel 9 (comprising laser fiber 20 in FIG. 10C) to a fluid port 22 (not shown in FIG. 10C). Suction connection 21 isolates working channel 9 from interstitial space 16. In the embodiments shown in FIG. 10C, fluid port 22 is not in fluid communication with interstitial space 16.

In some embodiments, the internal components of a device with an interstitial space are sealed to allow irrigation to flow through the device without damaging any internal components of the device. For example, in some embodiments, a fluid port in fluid communication with the interstitial space is utilized. In some embodiments, the device comprises a fluid seal for inserting wires and/or sensors between the fluid port and the outer diameter of any working channels 4. The seal prevents fluid in the interstitial space from flowing into the handle of the endoscope/ureteroscope device and focuses the fluid pressure toward any interstitial flow opening(s).

In some embodiments, the ureteroscopes described herein are provided as part of a system. An exemplary system is shown in FIG. 9. Referring to FIG. 9, shown is a system comprising ureteroscope tip 26, temperature and/or pressure sensor 27, ureteroscope handle 28, suction port distal end 29, and working channel distal end 30. In some embodiments, the system includes an irrigation delivery system, laser, and camera (not shown in FIG. 9). In some embodiments, the system includes a mechanism to articulate the ureteroscope tip 26. In some embodiments, systems include a component configured to move suction and/or laser/irrigation delivery systems between symmetrical working channels. In some embodiments, the handle/system includes a mechanism to linearly translate the laser forward and backward with respect to the long axis of the device. This can be accomplished through a manual sliding mechanism or other means. This can be useful to unclog the device when suction is applied through working channel 9 with laser included. If a clog occurs, it will tend to occur near the entrance of the tip. By moving the laser fiber backward then forward (by about an inch), one can quickly clear any stone fragments that may have clogged or partially clogged working channel 9.

In some embodiments, the technology comprises use of a computer system. In some embodiments, computer systems comprising a computer processor, computer, and user interface (e.g., monitor, smart phone, tablet, or smart watch) is used to operate one or more functions of the system, including but not limited to, the clog detection and removal component and the irrigation and suction calibration component. For example, in some embodiments, as described above, the computer system detects and removes clogs in an automated fashion. In addition, in some embodiments, the computer system automatically calibrates and maintains optimal irrigation flow rates.

In some embodiments, a computer system comprises a bus or other component configured to communicate information and a hardware processor coupled with the bus for processing information. In some embodiments, the hardware processor is a general-purpose microprocessor. In some embodiments, the computer system comprises a main memory, such as a random access memory (RAM) or other dynamic storage device, coupled to the bus for storing information and instructions to be executed by the processor. In some embodiments, the main memory is used for storing temporary variables or other intermediate information during execution of instructions to be executed by the processor. In some embodiments, instructions (e.g., stored in non-transitory storage media accessible to the processor) are provided to a computer system to provide a special-purpose machine that is customized to perform the operations specified in the instructions. In some embodiments, the computer system comprises a read only memory or other static storage device coupled to the bus for storing static information and instructions for the processor. In some embodiments, a storage device, such as a magnetic disk, optical disk, or solid-state drive, is provided and coupled to the bus for storing information and instructions. In some embodiments, the computer system is coupled by the bus to a display, such as a cathode ray tube (CRT), liquid crystal display (LCD), or other display technology known in the art, for displaying information to a computer user. In some embodiments, an input device (e.g., including alphanumeric and other keys) is coupled to the bus for communicating information and command selections to the processor. In some embodiments, other types of user input devices that find use for cursor control include, e.g., a mouse, a trackball, or cursor direction keys for communicating direction information and command selections to the processor and for controlling cursor movement on the display. Input devices typically have two degrees of freedom in two axes, a first axis (e.g., x) and a second axis (e.g., y), that allows the device to specify positions in a plane.

In some embodiments, the computer system implements embodiments of the technology described herein using customized hard-wired logic, one or more ASICs or FPGAs, firmware, and/or program logic that, in combination with the computer system, causes or programs the computer system to be a special-purpose machine. In some embodiments, methods described herein are performed by a computer system in response to a processor executing one or more sequences of one or more instructions contained in main memory. In some embodiments, instructions are read into main memory from another storage medium, such as a storage device. In some embodiments, execution of the sequences of instructions contained in main memory causes a processor to perform the process steps described herein. In some embodiments, hard-wired circuitry is used in place of or in combination with software instructions.

As used herein, the term “storage media” refers to any non-transitory media that store data and/or instructions that cause a machine to operate in a specific fashion. Such storage media may include non-volatile media and/or volatile media. Non-volatile media includes, for example, storage devices (e.g., optical disks, magnetic disks, or solid-state drives). Volatile media includes dynamic memory, such as main memory. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, or any other memory chip or cartridge. Storage media is distinct from but may be used in conjunction with transmission media. Transmission media participates in transferring information between storage media. For example, transmission media includes coaxial cables, copper wire and fiber optics, including the wires that include a bus. Transmission media can also take the form of acoustic or light waves, such as those generated during radio-wave (e.g., IEEE 802.11) and infra-red data communications. Transmission media also includes the Internet, WAN, and LAN.

In some embodiments, various forms of media carry one or more sequences of one or more instructions to a processor for execution. For example, the instructions may initially be carried on a magnetic disk or solid-state drive of a remote computer. The remote computer can load the instructions into its dynamic memory and send the instructions over a transmission medium. A local computer system can receive the data on the transmission medium and appropriate circuitry can place the data on a bus. The bus carries the data to main memory, from which a processor retrieves and executes the instructions. The instructions received by main memory may optionally be stored on a storage device either before or after execution by the processor. In some embodiments, a computer system comprises a communication interface coupled to the bus. In some embodiments, a communication interface provides a two-way data communication coupling to a network link that is connected to a local network. For example, a communication interface may be an integrated services digital network (ISDN) card, cable modem, satellite modem, ethernet card, wireless radio, or a modem to provide a data communication connection to a corresponding type of telephone line. As another example, a communication interface may be a local area network (LAN) card to provide a data communication connection to a compatible LAN. Wireless links may also be implemented. In any such implementation, the communication interface sends and receives electrical, electromagnetic, or optical signals that carry digital data streams representing various types of information.

In some embodiments, a network link provides data communication through one or more networks to other data devices. For example, a network link may provide a connection through a local network to a host computer or to data equipment operated by an Internet Service Provider (ISP). An ISP in turn provides data communication services through the world wide packet data communication network now commonly referred to as the “Internet”. A local network and the Internet both use electrical, electromagnetic, or optical signals that carry digital data streams. The signals through the various networks and the signals on the network link and through the communication interface, which carry the digital data to and from a computer system, are example forms of transmission media.

In some embodiments, the computer system sends messages and receives data, including program code, through the network(s), network link, and communication interfaces. For example, a server can transmit a requested code for an application program through the Internet, ISP, local network, and communication interface. In some embodiments, the received code is executed by a processor as it is received and/or stored in a storage device or other non-volatile storage for later execution.

In some embodiments, any of these hardware components are provided as a virtual component using emulation and/or cloud computing. Accordingly, as used herein, the term “hardware” and components of hardware described herein also refer to local or remote physical hardware or hardware and/or hardware components that are provided by emulation on as one or a plurality of virtual machines in a cloud environment.

In various embodiments, aspects of the described subject matter may be implemented by computer-executable instructions, e.g., stored on one or more computer-readable storage media described herein and/or known in the art. Computer-executable instructions may be implemented using any various types of suitable programming and/or markup languages such as: Extensible Application Markup Language (XAML), XML, XBL HTML, XHTML, XSLT, XMLHttpRequestObject, CSS, Document Object Model (DOM), JAVA, JavaScript, JavaScript Object Notation (JSON), Jscript, ECMAScript, Ajax, FLASH, SILVERLIGHT, VISUAL BASIC (VB), VB Script, PHP, ASP, SHOCKWAVE, Python, PERL, C, Objective-C, C++, C#/.net, Swift, SmallTalk, and/or others.

As used herein, the term “user interface” (UI) refers to a program interface that utilizes displayed graphical information to allow a user to control and/or operate a software application (e.g., a web application (e.g., a web page)), for example, by a pointer and/or a pointing device. A pointer may refer to a cursor, arrow, or other symbol appearing on a display and may be moved or controlled with a pointing device to select objects, populate fields, input commands, etc. via the UI. A pointing device may refer to any object and/or device used to control a cursor and/or arrow, to select objects, to populate fields, or to input information such as commands and/or drop-down menu options, for example, via a UI of the web application. Such pointing devices may include, for example, a mouse, a trackball, a track pad, a track stick, a keyboard, a stylus, a digitizing tablet, a digital pen, a fingertip in combination with a touch screen, etc. A cursor may refer to a symbol or pointer where an input selection or actuation may be made with respect to a region in a UI.

In some embodiments, the systems described herein find use in ablating kidney stones. For example, in some embodiments the ureteroscope of a system described herein (e.g., comprising clog detection and removal components and/or suction and/or irrigation flowrate calibration components) is inserted in the ureter of a subject and advanced to the vicinity of a stone. Once a stone is visualized, laser ablation, in combination with clog detection removal and irrigation and suction (e.g., using the irrigation and suction calibration systems described herein) is performed. Once the stone has been ablated and debris fragments and stone dust have been satisfactorily removed via suction, the ureteroscope is removed.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A system, comprising:
a) an endoscope;
b) a suction component and/or an irrigation component; and
c) a clog detection and removal component.

2. The system of claim 1, wherein said suction component and said irrigation component are pressure and/or flowrate controlled.

3. The system of claim 1, wherein said suction component comprises a volumetric peristaltic pump.

4. The system of claim 1, wherein said irrigation component is selected from the group consisting of a pressure chamber, a gravity bag, a pressure bag, a peristaltic pump, and a combination thereof.

5. The system of claim 1, wherein said irrigation component is configured to provide irrigation at a flowrate between 0-60 ml/minute.

6. (canceled)

7. The system of claim 1, wherein said clog detection and removal component comprises one or more clog detectors.

8. The system of claim 7, wherein said clog detectors are selected from the group consisting of a weight scale or load cell that measures the rate change in weight of a collection canister, a flowmeter in a part of an aspiration tube, an ultrasonic wave flowmeter, a photo resistor shining light through said aspiration tubing, and a pressure monitor.

9. The system of claims 7, wherein said clog detectors are located downstream from said suction component, upstream of said suction component, within a fluid collection canister, below said fluid collection canister, before or after the volumetric pump, within the fluid collection canister, below the collection canister, within said endoscope, or a combination thereof.

10. The system of claim 7, wherein said clog detection and removal component further comprises a clog removal component.

11. The system of claim 10, wherein said clog removal component is configured to respond to the presence of a clog by taking one or more actions selected from the group consisting of reducing the irrigation pressure/flowrate to minimize a rise in intra renal pressure; increasing the irrigation pressure/flowrate; momentarily stopping/reversing the flow of the suction channel to unclog any fragments that clogged the tip of the device; alarming a user, and vibrating or actuating said endoscope to shake loose the clog.

12. The system of claim 1, wherein said system monitors the number of clogs and/or the severity of said clogs over time.

13. The system of claim 12, wherein if the number and/or severity of the clogs is above a preset threshold, the system alerts a user and/or decreases the normal suction flowrate.

14. The system of claim 12, wherein if the number and/or severity of the clogs is below a preset threshold, the system increases the normal suction flowrate.

15. The system of claim 7, wherein said clog detection and removal component is automated or manually controlled.

16. (canceled)

17. The system of claim 1, wherein said system further comprises an irrigation and suction flowrate calibration component.

18. The system of claim 17, wherein said irrigation and suction flowrate calibration component is configured to a) measure intrarenal pressure during irrigation and/or suction; b) extrapolate the intrarenal pressure to determine the anticipated steady state pressure; and c) predict optimal suction and/or irrigation parameters that maintain a target intrarenal pressure for said system.

19. The system of claim 18, wherein said irrigation and suction flowrate calibration component is further configured to adjust input pressure and/or flowrate to maintain said intrarenal pressure at said optimal steady state intrarenal pressure during use of said system.

20. The system of claim 17, wherein said irrigation and suction flowrate calibration component comprises a pressure sensor.

21-27. (canceled)

28. The system of claim 1, wherein said endoscope is an endoscopic device, comprising:
an outer housing surrounding an interstitial space, wherein said endoscopic device comprises at least one interstitial flow opening(s) configured to deliver fluid or suction through said interstitial space.

29. A system, comprising:
a) an endoscope comprising a distal end, said distal end comprising: a) a first channel configured for delivery of fluid; and b) a second channel configured to remove fluid via suction, wherein the exit of said second channel comprises a suction port;
b) a suction component and/or an irrigation component; and c) an irrigation and suction flowrate calibration component configured to i) measure intrarenal pressure during irrigation and/or suction; ii) extrapolate the intrarenal pressure to determine the anticipated steady state pressure; and iii) predict optimal suction and/or irrigation parameters that maintain a target intrarenal pressure for said system.

30. (canceled)

31. A method of ablating a kidney stone, comprising:
a) introducing the endoscopic device of claim 1 into the ureter of a subject;
b) advancing the endoscopic device to a kidney stone while said flowrate controlled suction component, said irrigation component, and said clog detection and removal component are active; and
c) ablating said stone using said endoscopic device.

32-36. (canceled)
